(19) United States
(12) Patent Application Publication
(10) Pub. No.: US 2013/0102826 A1
(43) Pub. Date: Apr. 25, 2013

(54) Systems And Methods For Generating Alpha Olefin Oligomers

(71) Applicants: James R. Lattner, Laporte, TX (US); Michael W. Weber, Houston, TX (US)

(72) Inventors: James R. Lattner, Laporte, TX (US); Michael W. Weber, Houston, TX (US)

(21) Appl. No.: 13/448,862

(22) Filed: Apr. 17, 2012

Related U.S. Application Data
(60) Provisional application No. 61/489,424, filed on May 24, 2011.

(51) Int. Cl. C07C 2/08

(52) U.S. Cl. CPC C07C 2/08
USPC 585/510; 422/131

(57) ABSTRACT

Methods for preparing selected oligomers from monomers utilize systems of equipment adapted to provide desired compositions in various streams. Representative equipment of an oligomerization system includes an oligomer synthesis reactor and, optionally, a gas/liquid phase separation system. A monomer feed stream and a catalyst feed stream are directed to the oligomer synthesis reactor. The reactor produces a vapor phase effluent and a liquid phase effluent. The selected oligomer product is withdrawn from the vapor phase effluent. When the gas/liquid phase separation system is included, it is adapted to form a first recycle stream and a separator product stream from the vapor phase effluent. The separator product stream includes the desired oligomer product. Additional equipment may be utilized to further refine the vapor phase effluent and/or the final product stream.

### CROSS-REFERENCE TO RELATED APPLICATIONS

This application is related to Provisional Application No. 61/489,424 filed May 24, 2011, the disclosure of which is fully incorporated herein by reference.

### FIELD

The present disclosure relates to chemical reactions, processes for controlling chemical reactions, separation processes, and systems for performing such processes. The present disclosure further relates to systems and methods for utilizing one or more streams within such systems and processes to target generation of particular oligomers from monomers using catalyst systems.

### BACKGROUND

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

1-Hexene can be produced in high selectivity via ethylene trimerization using homogeneous, single-site chromium catalyst systems, activated by a molar excess of alkyl aluminums such as methyl alumoxane (MAO) and modified methyl alumoxane (MMAO). 1-Hexene has many potential uses, one of which is as a comonomer in higher order polyolefin reactions. The reactions to form higher order polyolefins, such as polyethylenes of varying grades, is dependent on the comonomer introduced into the reaction. As the demand for polyethylenes, and particularly for polyethylene grades that require inclusion of one or more comonomer, the demand for 1-hexene and other select comonomers continues to increase.

The trimerization reaction of ethylene to 1-hexene represents one method of manufacturing desired oligomer product as needed. Similarly, 1-octene and other desired oligomer products can be produced in high selectivity via ethylene oligomerization using homogeneous chromium catalyst systems activated by an appropriate aluminum compound. Such selective oligomerization reactions have been performed for many years with many, many optimization efforts. Exemplary past processes descriptive of the reaction chemistry can be found at least in U.S. Pat. No. 7,157,612, and in International Patent Publication Nos. WO2007/092136 and WO2009/060343, each of which is incorporated herein by reference in its entirety for all purposes. One of the major challenges associated with the selective oligomerization of ethylene (or other olefins) is the control of the reaction to maximize production rates while maintaining selectivity to the desired oligomer and maximizing catalyst utilization rates.

In conventional oligomerization systems 10, such as illustrated in FIG. 1, the desired oligomer product is withdrawn from the reactor 12 as a liquid mixture, including the desired oligomer, catalyst, diluent from the reactor, and still unreacted monomer. The liquid mixture in the conventional reactor bottoms stream 14 undergoes several processes, shown generally as process boxes 16, 18, to isolate a product stream 20 in which the desired oligomer is relatively concentrated. In the illustration of FIG. 1, the reactor bottoms stream is separated by a first process 16 to isolate byproducts and other waste materials in a purge stream 22 and to provide an enriched stream 24. The purge stream 22 may include components such as used catalyst and reaction by-products, like longer chain polymers. The enriched stream 24 illustrated in FIG. 1 conventionally comprises the desired oligomer, unreacted monomer, and diluent. Continuing with the description of the representative oligomer synthesis reactor system 10 of FIG. 1, the enriched stream 24 is then passed through a second process 18 to further separate the desired oligomer from the diluent and the unreacted monomer. As illustrated, process 18 produces a diluent recycle stream 26 and the product stream 20. The diluent recycle stream 26 generally comprises diluent and unreacted monomer, with preferably very little of the desired oligomer.

FIG. 1 illustrates further aspects of an oligomer synthesis reactor system 10. As can be seen, the diluent recycle stream 26 is directed to a mixer 28 and combined with a vapor recycle stream 30. The mixer 28 may include a cooler or other heat exchange facility to change the properties of the combined recycle stream 32. The combined recycle stream, a monomer make-up stream 34, and a catalyst feed 36 are each illustrated as inputs to the reactor 12. Additionally, the reactor system 10 is provided with evaporative cooling features, including a reflux of condensed portions of the gaseous reactor top stream 38. As is well understood, the oligomerization reaction is exothermic and temperature control is a critical aspect of successful operations. Evaporative cooling operations remove heat from the reaction by allowing a portion of the reaction solution to evaporate. As suggested previously, the inputs to the reactor, such as the mixed recycle stream 32 may be temperature controlled to further regulate the temperature inside the reactor 12. In the illustration of FIG. 1, and as previously disclosed in WO 2007/092136, reactor systems 10 including evaporative cooling features may include a chiller 40 and a separator 42 to provide a liquid reflux stream 44 and a vapor recycle stream 30. While the separator will not necessary provide a perfect split of the heavy and light components in the overhead stream 38, the heavier components will be more concentrated in the separator bottoms stream 44, and the lighter components will be more concentrated in the separator overhead stream 30.

As is well understood, oligomer synthesis reactor systems and operations are optimally designed and operated to maximize production rates of the desired oligomer while optimizing/balancing other objectives, including: 1) maximizing catalyst utilization rates; 2) maximizing reaction selectivity, which can also be stated as minimizing the production of unwanted oligomers, such as higher order oligomers (referred to generally herein as “byproduct polymers”) resulting from continued oligomerization of the desired oligomer; and 3) minimizing overall capital and operating costs. There are at least three recognized approaches to increasing production rates: 1) increasing the flow rates through the reactor, resulting in a shorter residence time; 2) increasing the reactor size to allow for higher flow rates without decreasing the residence time; and 3) increasing the residence time in the reactor to maximize the catalyst utilization, which generally reduces the reaction selectivity. As can be readily understood, each of these options has inherent disadvantages. Increasing the reactor size incurs significant capital expenditures, which changes the economics of an entire project. Significantly, the option of increasing the reactor size is even less available or more expensive in the context of a retro-fit or upgrade of an existing system. The other two options both relate to the rate at which streams enter and/or exit the reactor and will be discussed in more detail below.

As can be seen in FIG. 1, the desired oligomer exits the reactor 12 en route to a product stream through the reactor liquid bottoms stream 14. Because the catalyst is also a component in the liquid bottoms stream 14, the rate at which the oligomer is withdrawn from the reactor is proportional to the rate at which the catalyst and unreacted monomer are withdrawn. Accordingly, any change in residence time, such as by changing flow rates, will directly affect oligomer selectivity and catalyst utilization. If the oligomer production rate is increased by increasing the flow rate through the reactor, the catalyst is also flowed through the reactor at a higher rate. As the catalyst is killed or deactivated after exiting the reactor, its recycle is impractical. In the event that the catalyst's natural life is timed to correspond to the decreased residence time in the reactor, the higher catalyst flow rate will not negatively impact the economics of the operation. However, many modern catalyst systems are known to have active lives that are longer than even the standard reactor residence times. In other implementations, some catalyst systems are known to have induction periods requiring a minimum residence time for activation of the catalyst, which effectively establishes a maximum flow rate. Regardless of the catalyst system selected, a shorter residence time resulting from increased flow rates in the pursuit of increased production rates, necessarily results in decreased catalyst utilization rates. Whereas the catalyst is generally a specialty product rather than a commodity product, it is often the most expensive feedstock in the oligomer synthesis reaction. Accordingly, while increasing the production rate may be desirable, it is not generally accepted at the expense of the catalyst.

When capital costs requirements and catalyst utilization requirements preclude increased production through the methods described above, the application of conventional understanding is to increase the product concentration in the reactor to increase the concentration of desired oligomer product in the bottoms stream 14. However, this approach has its own limitations. By increasing the concentration of the desired oligomer product in the reactor, the rate of re-incorporation of the oligomer also increases, which results in an increased production of the byproduct polymers. In effect, the catalyst utilization increases but its selectivity decreases.

Accordingly, it can be seen that conventionally accepted methods of increasing production rates of desired oligomer products are accompanied by undesirable consequences. In the past, operators have been forced to optimize operations balancing the positive and negative aspects of each strategy and designing systems and methods focused on a currently perceived sweet spot. Unfortunately, with one of the strategies intimately linked to a one time event with a fixed cost (reactor construction); some flexibility is necessarily lost after the reactor system is first constructed. This limits the operator's ability to adapt to changing costs and capabilities of feedstock, such as the catalysts.

The foregoing discussion of need in the art is intended to be representative rather than exhaustive. Multiple opportunities remain for improvement in the oligomer synthesis reactor systems and methods.

### SUMMARY

The present disclosure provides methods for preparing oligomers from monomers. In some implementations, the method comprises: 1) feeding a monomer feedstream, a catalyst feedstream, and a diluent to the oligomer synthesis reactor; 3) oligomerizing the monomer in the presence of the catalyst in the reactor to produce a vapor phase effluent and a liquid phase effluent; and 4) utilizing at least a portion of the vapor phase effluent from the reactor as an oligomer product stream. The reaction is carried out under conditions to produce an oligomer product, such as 1-hexene and/or 1-octene. The vapor phase effluent comprises unreacted monomer, oligomer product, and diluent. The liquid phase effluent comprises catalyst and diluent.

In some implementations, the methods may further comprise providing a gas/liquid phase separation system adapted to receive the vapor phase effluent. In implementations including a gas/liquid phase separation system, passing the vapor phase effluent through the gas/liquid phase separation system forms a first recycle stream and a separator product stream. The first recycle stream comprises primarily diluent and un-reacted monomer from the vapor phase effluent. The separator product stream comprises a majority of the oligomer in the vapor phase effluent. Of course, the first recycle stream may include some minor portion of the oligomer in the vapor phase effluent and the separator product stream may include some remaining diluent and un-reacted monomer. The configuration of the gas/liquid phase separation system may be selected based at least in part on the desired degree of separation to be accomplished. Any conventional gas/liquid phase separation system may be utilized. Regardless of the specific gas/liquid phase separation system utilized, the separator product stream has a higher concentration of the oligomer produced than the vapor phase effluent. Such methods include utilizing at least a portion of the separator product stream as the oligomer product stream.

Additionally, some implementations of the present methods may include providing a diluent recovery system adapted to separate the oligomer from diluent and unreacted monomer in the separator product stream. The separator product stream may be passed through the diluent recovery system to form a concentrated oligomer product stream and a second recycle stream or a diluent recycle stream. The second recycle stream recycles diluent and unreacted monomer to the oligomer synthesis reactor. The concentrated oligomer product stream may be utilized as the oligomer product stream.

Still additionally, some implementations of the present methods may include providing a byproduct separation system adapted to receive the liquid phase effluent from the reactor. The methods may include passing the liquid phase effluent through the byproduct separation system to produce an oligomer-rich stream and a purge stream. The oligomer-rich stream may include diluent, un-reacted monomer, and oligomer. The purge stream may include catalyst, byproduct polymers, some diluent and minimal amounts of oligomer. The methods may further include passing the oligomer-rich stream through the diluent recovery system described above to separate oligomer from diluent and unreacted monomer in the oligomer-rich stream. The separated oligomer may be added to the concentrated oligomer product stream described above; the diluent and unreacted monomer is recycled to the oligomer synthesis reactor.

While the methods of the present disclosure may be implemented using a wide diversity of systems and equipment, the present disclosure also provides examples of systems adapted and suited for the implementation of the present methods. In some respects, the systems of the present disclosure will be described in generic fashion allowing one of skill in the art to select from a diversity of systems adapted to provide the recited function. For example, the present systems include an oligomer synthesis reactor and may include a gas/liquid phase separation system. It can be readily understood that an oligomer synthesis reactor incorporates thousands of design decisions embodied as hundreds of mechanical and electrical parts. It is not the ambition of the present disclosure to describe such reactors in the level of detail required to build and operate a functioning unit because one of ordinary skill will have the skills required to do so without undue experimentation. Similarly, one of ordinary skill in the industry is well aware of multiple categories of gas/liquid separation systems and can design hundreds of such systems in each category. For example, a simple chiller and flash drum combination can be implemented in hundreds of manners. Similarly, a more complicated gas/liquid phase separation system, like a distillation column, may have thousands of implementations at the ready disposal of one of ordinary skill.

Despite the potentially conventional nature of some individual component parts of the present systems, it is the arrangement and functionality of the component parts that renders the systems of the present disclosure inventive. Stated otherwise, the inventive systems of the present disclosure are believed to provide novel arrangements of component equipment parts that collectively provide a system not heretofore described. As one example, the present systems include an oligomerization system for preparing oligomers from monomers. The oligomerization system comprises an oligomer synthesis reactor. The reactor is adapted to receive a monomer, a catalyst, and a diluent. The reactor is further adapted to oligomerize, such as trimerize, the monomer in the presence of the catalyst to produce an oligomer product. The reactor produces a vapor phase effluent and a liquid phase effluent. The vapor phase effluent comprises unreacted monomer, oligomer, and diluent. The liquid phase effluent comprises catalyst and diluent. In systems of the present disclosure, at least a portion of the vapor phase effluent is utilized as an oligomer product stream.

Additionally or alternatively, the systems of the present disclosure may further include a gas/liquid phase separation system adapted to receive the vapor phase effluent. The gas/liquid phase separation system is configured to form a first recycle stream and a separator product stream. In such implementations, there may also be included a recycle loop adapted to recycle the first recycle stream to the oligomer synthesis reactor. The first recycle stream comprises diluent and unreacted monomer. The separator product stream comprises a majority portion of the oligomer product in the vapor phase effluent. At least a portion of the separator product stream is utilized as the oligomer product stream.

Still additionally or alternatively, the systems of the present disclosure may further include a diluent recovery system adapted to receive the separator product stream. The diluent recovery system may be adapted to separate oligomer product from diluent and unreacted monomer to form a concentrated oligomer product stream and a second recycle stream. Oligomerization systems that include a diluent recovery system may additionally include an additional recycle loop adapted to recycle the second recycle stream, comprising diluent and unreacted monomer, to the oligomer synthesis reactor. The concentrated oligomer product stream comprises a majority portion of the oligomer product in the separator product stream. The concentrated oligomer product stream may be utilized as the oligomer product stream.

Still additionally, the oligomerization systems including the diluent recovery systems may further include a byproduct separation system. The byproduct separation system is adapted to receive and process the liquid phase effluent to produce an oligomer-rich stream and a purge stream. The reactor systems may further include a purge system adapted to discharge the purge stream from the oligomerization system. The oligomer-rich stream is directed to the diluent recovery system. The diluent recovery system is adapted to receive both the separator product stream and the oligomer-rich stream and to form a concentrated oligomer product stream therefrom.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE FIGURES

The foregoing and other advantages of the present technique may become apparent upon reading the following detailed description and upon reference to the drawings in which:

FIG. 1 is a schematic flow diagram of a representative, conventional oligomerization system including a oligomer synthesis reactor;

FIG. 2 is a schematic flow diagram of an oligomerization system according to the present disclosure, including an optional gas/liquid phase separation system;

FIG. 2A is a schematic illustration of a representative gas/liquid phase separation system that may be implemented in the system of FIG. 2;

FIG. 2B is a schematic illustration of an alternative gas/liquid phase separation system that may be implemented in the system of FIG. 2;

FIG. 3 is a schematic illustration of an oligomerization system according to the present disclosure, including an optional gas/liquid phase separation system;

FIG. 4 is a schematic illustration of an oligomerization system including various, optional post-processing equipment; and

FIG. 5 is a graph illustrative of the impact of diluent selection on oligomerization processes.

While the technologies of the present disclosure are susceptible to various modifications and alternative forms, specific exemplary embodiments thereof are shown in the drawings and are herein described in detail. It should be understood, however, that the description herein of specific exemplary embodiments is not intended to limit the disclosure to the particular forms disclosed herein, but on the contrary, this disclosure is to cover all modifications and equivalents of the technologies defined by the appended claims. It should also be understood that the drawings are not necessarily to scale, emphasis instead being placed upon clearly illustrating principles of exemplary embodiments of the present invention. Moreover, certain dimensions may be exaggerated to help visually convey such principles. For the purposes of clarity, not every component is labeled in every figure, nor is every component of each embodiment of the invention shown where illustration is not necessary to allow those of ordinary skill in the art to understand the invention.

### DETAILED DESCRIPTION

In the following detailed description section, specific aspects of the present inventions are described in connection with preferred implementations. However, to the extent that the following description is specific to a particular embodiment or implementation or a particular use of the present inventions, this is intended to be for exemplary purposes only and simply provides a description of the exemplary embodiments. Accordingly, the invention is not limited to the specific embodiments described below, but rather, it includes all alternatives, modifications, and equivalents falling within the true spirit and scope of the appended claims.

In the interest of clarity, not all features of an actual implementation are described in this disclosure. It will of course be appreciated that in the development of any such actual embodiment, numerous implementation-specific decisions must be made to achieve the developer's or operator's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of the present disclosure.

In an effort to provide clarity to the some of the terms used below, the context of certain terms is provided before embarking on the more detailed description of the present systems and methods. While several terms are here discussed, it should be noted that all terms used herein are intended to communicate their ordinary meaning unless a different meaning is specifically and explicitly applied herein. The indefinite articles “a” and “an” as used herein mean one or more when applied to any feature in embodiments of the present invention described in the specification and claims. The use of “a” and “an” does not limit the meaning to a single feature unless such a limit is specifically stated. As such, the terms “a” (or “an”), “one or more” and “at least one” can be used interchangeably herein. The adjective “any” means one, some, or all indiscriminately of whatever quantity.

Unless otherwise indicated, all numbers expressing quantities of ingredients, properties such as molecular weight, concentrations, reaction conditions, temperatures, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in the light of the number of reported significant digits and by applying ordinary rounding techniques. As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly.”

The term “and/or” placed between a first entity and a second entity means one of (1) the first entity; (2) the second entity; and (3) the first entity and the second entity. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements, other than B); in another embodiment, to B only (optionally including elements other than A); in yet another embodiment, to both A and B (optionally including other elements).

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements). The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together, each of which may include additional non-enumerated elements.

Reference throughout this specification to “one embodiment”, “one implementation”, “an embodiment” or “an implementation” means that a particular feature, structure, or characteristic described in connection with the embodiment and/or implementation may be included in at least one implementation and/or embodiment of claimed subject matter. Thus, the appearances of the phrase “in one embodiment”, “an embodiment”, “in one implementation” or “a feature” in various places throughout this specification are not necessarily all referring to the same embodiment and/or implementation. Furthermore, the particular features, structures, or characteristics may be combined in one or more implementations and/or embodiments.

Exemplary systems and methods may be better appreciated with reference to flow diagrams. While for purposes of simplicity of explanation, the illustrated systems and methods are shown and described as a series of blocks, it is to be appreciated that the systems and methods are not limited by the order of the blocks, as in different embodiments some blocks may occur in different orders and/or concurrently with other blocks from that shown and described. Moreover, less than all the illustrated blocks may be required to implement an exemplary system and/or method. In some examples, blocks may be combined, separated into multiple components, may employ additional, not illustrated blocks, and so on.

The terms “predominantly,” “primarily,” “principally,” and “in major portion,” when used to describe the presence of a particular component of a fluid stream, mean that the fluid stream comprises at least 50 mole percent of the stated component. As will be understood, these terms may similarly refer to at least 50 weight percent, depending on the particular context of their usage, which will be understood by one of ordinary skill. For example, a “predominantly” methane stream, a “primarily” methane stream, a stream “principally” comprised of methane, or a stream comprised “in major portion” of methane each denote a stream comprising at least 50 mole percent methane. The inverse can be understood of phrases such as “a minor portion.” Similarly, terms such as the “the majority portion of” are intended to refer to greater than 50 mole percent of the particular component. For example, “the majority portion of the hexene from (or in)” refers to greater than 50 mol % (or weight percent) of the hexene from the stream.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and about 200, but also to include individual sizes such as 2, 3, 4, and sub-ranges such as 10 to 50, 20 to 100, etc.

The term “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition, or are included only in amounts which are small enough so as to have no measurable effect on the composition.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions, or examples, which are included to enable a person having ordinary skill in the art to make and use the inventions, when the information in this patent is combined with available information and technology.

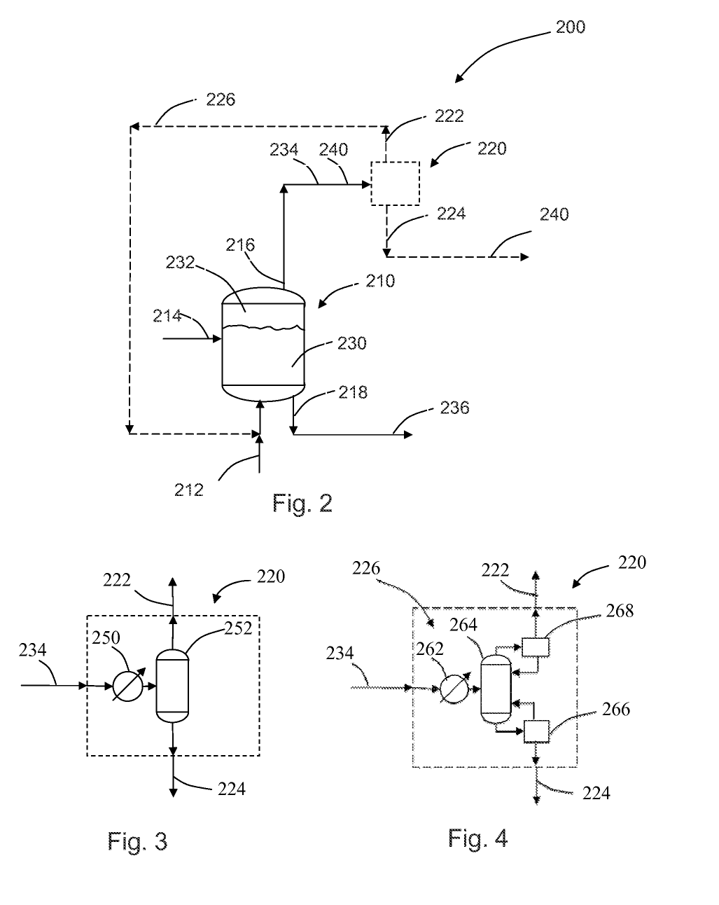

As introduced above, the present disclosure provides methods and systems for preparing select oligomers from monomers. FIG. 2 provides a representative schematic of a system adapted for implementation of the present methods. The equipment illustrated in FIG. 2 is intentionally generic due to the many implementation-specific variables that will affect the individual component equipment parts. Accordingly, FIG. 2 illustrates the relationship between the parts, to illustrate the methods, rather than the nuance of the specific parts. FIG. 2 illustrates an oligomerization system 200, which includes an oligomer synthesis reactor 210 and an optional gas/liquid phase separation system 220. As inputs to the reactor 210, FIG. 2 illustrates a monomer feed stream 212 and a catalyst feed stream 214. FIG. 2 further illustrates a vapor phase effluent 216 and a liquid phase effluent 218 exiting the reactor 210. As illustrated, the vapor phase effluent 216 is directed to the gas/liquid phase separation system 220, which is illustrated in dashed lines to indicate that it is optional in the systems of the present disclosure. The optional gas/liquid phase separation system 220 is illustrated as forming a first recycle stream 222 and a separator product stream 224. When the gas/liquid phase separation system 220 is included in the oligomerization system 200, the first recycle stream 222 may be recycled through a recycle loop 226 to the oligomer synthesis reactor 210. While the recycle loop 226 is illustrated as a simple recycle line, it should be understood the various conventional processes may be implemented on the recycle loop 226, such as mixing, heat exchange, compression, etc., before directing the first recycle stream back to the reactor 210.

With the primary components of the oligomerization system 200 described, specifics of certain components will now be described for clarity. The oligomer synthesis reactor 210 may be any suitable reactor configuration, which may be selected based on factors such as catalyst systems and monomers being used and oligomer product being produced. For the purpose of the present disclosure, the reactor 210 is adapted to include a liquid phase region 230 and a vapor phase region 232. As can be expected, the vapor phase region 232 is above the liquid phase region 230. The feed streams to the reactor 210, such as the monomer feed stream 212, the catalyst feed stream 214, and the first recycle stream 226, enter the reactor in the liquid phase region 230, regardless of the state of the materials in the stream. The oligomerization reaction occurring inside the reactor 210 generates heat (i.e., the reaction is exothermic). Accordingly, compositions in the reactor in the liquid phase, and having a boiling point below the internal temperature of the reactor will be evaporated and flow into the vapor phase region 232.

As has been described in prior patent applications, which have been incorporated herein by reference above, the reaction conditions in the reactor 210 may be controlled to maintain a desired temperature range within the reactor 210 by evaporative cooling. For example, the evaporation of the liquid phase and evacuation of the resultant vapor phase, or portions thereof, may withdraw sufficient energy of vaporization from the reactor to maintain a desired temperature. One exemplary implementation may maintain a desired temperature range by introducing excess monomer in order to maintain a specific rate of evaporation. For example, the reaction temperature may be maintained between about 50° C. and about 150° C. while maintaining the reaction pressure between about 150 psi (10.5 kg/cm2) to about 900 psi (63.3 kg/cm2). Additionally or alternatively, the temperature and/or the pressure may be controlled or regulated by other means, such as through the use of cooling equipment or pressurization equipment, within the reactor and/or on one or more of the feed streams. Reaction conditions selected to provide evaporative cooling with concurrent condensation on walls of the reactor 210 or other equipment has been found to provide anti-fouling benefits.

As can be well understood by those of skill in the art, the monomer in the reactor 210 is adapted to react in the presence of the catalyst to form an oligomer, having two or more monomers bonded together. Depending on the monomer and/or catalyst selected and the reaction conditions maintained in the reactor, the present systems and methods may be adapted to oligomerize the monomer into any number of possible oligomers. In exemplary implementations, the monomer feed may be ethylene. Ethylene may be oligomerized to form butene (dimerization), hexene (trimerization), octene, dicene, and higher-order oligomers. In some implementations the catalyst may selectively oligomerize the monomer to a desired oligomer, such as for use as a desired oligomer product. The selectivity of the catalyst may depend on multiple reaction conditions, including the concentration of monomer and oligomers in the reactor, the residence time of the monomer and oligomers in the reactor, temperature, etc. For the purposes of the present disclosure, any suitable catalyst system and set of reaction conditions may be utilized. Preferably, the oligomerization reaction will be conducted in a manner to maximize the selectivity of a desired oligomer product, such as 1-hexene.

Catalytic trimerization of ethylene to selectively produce 1-hexene is a well known reaction and process. Reactants, catalysts, diluents, reaction conditions, and reactor and separation apparatus configurations for such processes of this type are disclosed, for example, in U.S. Pat. Nos. 6,380,451 and 7,157,612 and in U.S. Patent Application Publication Nos. 2008/0058486; 2008/0182989; 2008/0188633; 2008/0200626; and 2008/0200743. All of these patent documents are incorporated herein by reference in their entirety.

In the methods disclosed herein, the principal reactant ethylene can be selectively trimerized to produce 1-hexene. Other olefin reactants, such as propylene, 1-butene, and 2-butene and the like, may also be trimerized as part of the reactor feed. Ethylene and/or the other olefins can also be dimerized or tetramerized as part of the reaction carried out in connection with the method herein.

Catalysts used to promote olefin (e.g., ethylene) oligomerization (e.g., trimerization) will generally comprise homogeneous, organometallic systems, such as single site, chromium catalyst systems. Such systems can comprise a chromium source in combination with a heterocyclic, di-aryl, or phosphorus compound such as a pyrrole, pyridyl or pyridyl-phosphino compound, along with an alkyl aluminum activator such as methyl alumoxane (MAO) or modified methyl alumoxane (MMAO). These and other suitable catalyst systems are well known in the industry. Suitable catalysts for use in the present systems and methods may be provided as a pre-formed catalyst system or one or more parts of the catalyst system may be provided to the reactor separately. For example, in some implementations the activator may be provided separately to the reactor for interaction with the remaining components of the catalyst system. Depending on a variety of factors, the catalysts utilized in the present systems and methods may be more or less active upon entering the reactor. The catalyst activity will increase as the chromium source, the alkyl aluminum activator and the monomer mix to form the active catalyst species. This induction period necessary for the catalyst system to reach its maximum activity can be ranged from 0.5 to 3 hours depending on the reactor conditions. As suggested above, the presence or absence of an induction period, and its relative duration, may affect the optimal residence time of the catalyst in the reactor.

The olefin reactant(s) and/or the catalyst system will generally be fed to the oligomerization reactor along with a suitable diluent. For purposes of this description, a “diluent” will be defined as the material added to the reactor feed, in addition to the catalyst and the ethylene or other olefin “reactant.” The diluents used herein will generally have a boiling point of from about −20° C. to 120° C. Such a diluent can typically be an inert hydrocarbon, such as C3-C6 normal and iso-paraffins, but can also be a cycloparaffin or aromatic compound. Olefins themselves can also be used as the reaction diluent. Olefins, however, are not preferred since, as noted, they can also serve as a reactant, depending on the catalyst system and conditions employed.

Having described the reactor 210 and the generalized conditions of the oligomer synthesis reaction carried out therein, the components of the feed streams into the reactor are somewhat self-evident. For example, the monomer feed stream 212 is a makeup stream to replace the monomer converted to the desired oligomer product in the reactor. Similarly, the catalyst feed stream 214 is provided to makeup or replace the catalyst that is lost during operation of the system. While a diluent feed stream is not illustrated in FIG. 2, it should be understood that diluent may be added together with either or both of the illustrated feed streams. Additionally, the diluent may be added through a separate feed stream. The catalyst feed stream 214 may provide components for catalyst systems and/or may provide pre-formed catalysts. As one example, the catalyst feed stream 214 may provide catalyst and activator separately or previously combined. As suggested by the discussion above, either or both of monomer feed stream 212 and catalyst feed stream 214 may be treated to provide a desired property to the reactor 210. For example, either or both streams may be cooled and/or pressurized. FIG. 2 further illustrates that first recycle stream 226 is directed back to the reactor 210. As will be better understood from the discussion below, the first recycle stream 226 may provide monomer, diluent, and/or other components to the reactor 210. In some implementations, the first recycle stream 226 may be mixed with or combined with the monomer feed stream 212 before entry into the reactor 210, as illustrated in FIG. 2, or may be fed directly into the reactor.

As illustrated in FIG. 2, the reactor 210 provides two distinct effluents: a vapor phase effluent 216 and a liquid phase effluent 218. Prior oligomer synthesis reactors, such as those described in prior patent applications previously incorporated herein by reference, have been designed to provide similar effluents. As can be understood from the foregoing description of the reactor 210 and the reactions occurring therein, the reactions and reaction conditions of the present disclosure may be implemented in any conventional manner that produces a vapor phase region 232 and a liquid phase region 230, from which a vapor phase effluent 216 and a liquid phase effluent 218 may be drawn, respectively. While the conventional systems and methods may appear similar to this point, it is the methods following the reactor that affect the overall oligomerization systems and methods, which has impacts back to the reactor and reaction conditions.

FIG. 2 illustrates that both the vapor phase effluent 216 and the liquid phase effluent 218 are carried away from the reactor 210. In conventional systems such as those described previously, the vapor phase in the reactor 210, when extracted, was cooled and recycled to the reactor for the cooling effects thereof. In such implementations, the only product stream carried away from the reactor 210 was the liquid phase effluent, which included the desired oligomer together with unreacted monomer, diluent, catalyst, and byproduct polymers. Separating the oligomer from this conventional product stream was its own source of multiple inventions to improve the overall system. However, as discussed above, the conventional practice of extracting the desired oligomer product together with the catalyst has inherent weaknesses, particularly where the catalyst active life and/or induction period suggests a desired catalyst residence time longer than the residence time of the desired oligomer product.

As illustrated in FIG. 2, the present systems and methods decouple the residence times of the catalyst and the desired oligomer product by treating the vapor phase effluent as a reactor product stream 234 rather than as a reactor reflux stream. The reactor product stream 234, which may be a treated or untreated vapor phase effluent 216, comprises the lighter components from the mixture in the reactor, such as desired oligomer product, unreacted monomer, and diluent. The precise composition of the reactor product stream 234 will depend on the selection of the diluent, the conversion rates of the monomer, the temperature, and pressure of the reactor, etc. It should be understood that not all of the desired oligomer product will exit the reactor in the vapor phase effluent. Regardless of the effective separation of the desired oligomer product (i.e., the split of oligomer product in the vapor phase effluent compared to the liquid phase effluent), the quantity of oligomer product removed from the oligomerization system by way of the reactor product stream is isolated from catalysts and from re-entry to the reactor, which precludes or substantially reduces the likelihood of individual oligomers, such as 1-hexene, being further oligomerized.

In some implementations, greater than 20% of the oligomer produced in the reactor 210 may exit through the vapor phase effluent 216. For example, between 20% and 99% of the produced oligomer may exit through the vapor phase effluent. The amount of produced oligomer exiting through one or the other effluent may be determined as the weight of oligomer in a particular effluent divided by the sum of the weights of oligomer in both streams. In preferred implementations, the diluent, reactor temperature, reactor pressure, and recycle rate (stream 226) may be adjusted to increase the percentage of desired oligomer product that exits the reactor through the vapor phase effluent 216, thereby becoming incorporated into the reactor product stream. In preferred implementations, the desired oligomer product present in the vapor phase effluent may be greater than about 40% of the total desired oligomer exiting the reactor. For example, 45%, 50%, or greater would be preferred.

In some exemplary implementations, the diluent selected may preferentially drive the desired oligomer product to the vapor phase effluent. For example, a low boiling point diluent with an affinity to the oligomer product may carry a greater portion of the oligomer product into the vapor phase effluent. Additionally or alternatively, a diluent having a higher boiling point than that of the oligomer product may allow a greater portion of the oligomer product to vaporize into the vapor phase region 232, thus becoming available to exit through the vapor phase effluent 216.

The liquid phase effluent 218 carries a portion of the liquids away from the reactor 210. The liquid phase effluent 218 may also be considered to be a reactor byproduct stream 236, which is intended to be disposed of or utilized for other purposes. The liquid phase effluent 218 may comprise diluent, catalyst, byproduct polymers, and other heavies from the reactor 210. It should be understood that in some implementations, the liquid phase effluent 218 may include more or less of each composition. Moreover, in some implementations, the liquid phase effluent 218 may be treated for various purposes. For example, the liquid phase effluent may be treated to deactivate the catalyst in the effluent to stop further oligomerization in the reactor byproduct stream 236. Additionally or alternatively, the liquid phase effluent may be treated to extract or separate desired oligomer product or other components from the effluent before it is discarded, as will be seen in the context of later Figures.

In some implementations, the recycle rate in stream 226 can be modified to control the amount of oligomer product recovered from the vapor phase. As the recycle rate is increased more oligomer product will be carried into the vapor phase effluent stream 216, thus becoming available to be recovered in the reactor product stream 234. In most implementations of the present systems and methods, the catalysts will be contained primarily in the liquid phase region 230. Accordingly, reaction of the monomer to form the desired oligomer occurs in the liquid phase region 230 where the monomer can interact with the catalyst. This reaction rate is not affected by the recycle rate in stream 226. The reaction rate is affected by the monomer concentration in the liquid which is fixed at a given temperature and pressure. A monomer recycle rate in excess of that needed to satisfy the reaction will serve to carry more oligomer product into the vapor phase as it is formed. In this way, the amount of product removed as a vapor can be controlled independent of other factors affecting reaction rate.

While the impact of the separate control on the vapor phase effluent 216 has been noted, it should also be noted that the distinct control over and separate treatment of the vapor phase effluent 216 and the liquid phase effluent 218 allows the reaction within the reactor 210 to be more precisely controlled. For example, the catalyst and other predominantly liquid phase components can be withdrawn from the reactor 210 at a first rate that will establish the residence time of the catalyst in the reactor, while the desired oligomer product and other predominantly vapor phase components may be withdrawn from the reactor 210 at a second rate. Accordingly, the residence time of the liquid phase (e.g., catalyst) may be controlled to optimize the catalyst utilization rate while the rate product is withdrawn through the vapor phase (e.g., oligomer product) may be controlled to reduce the concentration of product in the reactor and improve the selectivity of the oligomerization reaction.

FIG. 2 illustrates that in some implementations the vapor phase effluent 216 may provide a reactor product stream 234, some or all of which may be utilized as an oligomer product stream 240. The oligomer product stream 240 will be referenced several times and in several different contexts throughout this description. The oligomer product stream 240 refers to the stream to be utilized as the product of the oligomerization systems and methods herein. Accordingly, as will be seen, the vapor phase effluent 216, which is interchangeably referred to herein as the reactor product stream 234, may be the oligomer product stream 240. Still further, other streams downstream from the vapor phase effluent 216, such as after it has been further processed, may be utilized as the oligomer product stream. As used herein, the terms “utilization,” “utilizing,” and similar words, as they relate to use of a stream as the oligomer product stream 240, are intended to encompass multiple possible utilizations. For example, the stream utilized as the oligomer product stream 240 may be stored for later use or may be directed to another facility for incorporation into another process. Still additional representative utilizations may include flowing the oligomer product stream directly to another process at the same facility. Any conventional use of the oligomer product may be applied to the stream that is utilized as the oligomer product stream 240.

Additionally or alternatively, as illustrated in FIG. 2, the reactor product stream 234 may be directed to a gas/liquid phase separation system 220. The gas/liquid phase separation system 220 may be any suitable system and may include multiple component parts adapted to separate the gas from the liquid. As illustrated in FIG. 2, the gas portion of the reactor product stream 234 is recycled back to the reactor 210 as first recycle stream 222 while the liquid portion is withdrawn as the separator product stream 224, at least a portion of which may become the oligomer product stream 240. The first recycle stream 222 includes the diluent and the unreacted monomer from the reactor product stream 234, or at least a majority portion thereof. The separator product stream 224 includes a majority portion of the desired oligomer product found in the reactor product stream 234, as well as diluent and unreacted monomer.

The degree of separation effected in the gas/liquid phase separation system may be selected by the operator based on a variety of operating parameters. In some implementations it may be preferred to design the gas/liquid phase separation system to drive all, or substantially all, of the desired oligomer product into the separator product stream. For example, in order to reduce the possibility that desired product is recycled and potentially further oligomerized into byproduct polymer, it may be preferred to leave some diluent and unreacted monomer in the separator product stream to ensure that all of the desired oligomer product is driven to the separator product stream. In some implementations, 100% of the desired oligomer product in the reactor product stream 234 may be found in the separator product stream 224. Alternatively, the separator product stream 224 may include greater than about 75% of the desired oligomer product from the reactor product stream 234, preferably from about 75-100%, more preferably from about 85-100%, and still more preferably from about 90-100%.

As indicated above, the gas/liquid phase separation system 220 may comprise any suitable equipment for effecting the desired separation. FIGS. 2A and 2B schematically illustrate exemplary gas/liquid phase separation systems that may be incorporated into the oligomerization systems 200 of the present disclosure. FIG. 2A illustrates an exemplary gas/liquid phase separation system 220 comprising a heat exchanger 250 and a flash drum 252. Such an arrangement is conventional and could be designed in multiple manners by one of ordinary skill the art. In general, the incoming reactor product stream 234 enters the heat exchanger 250 and is cooled before entering the flash drum 252. The flash drum 252 allows the gas phase to separate from the liquid phase, which each exit the separator as described. The degree of cooling needed in the heat exchanger 250 and its configuration, as well as the configuration and operation of the flash drum 252, will depend at least in part on the composition and properties of the reactor product stream 234. Similarly, the configuration of these respective parts may depend on the degree of separation desired between the oligomer product and the diluent and monomer of the first recycle stream.

FIG. 2B illustrates yet another alternative implementation of a gas/liquid phase separation system 220. The gas/liquid phase separation system of FIG. 2B is a schematic distillation system 260, including exemplary components thereof. For example, the distillation system 260 includes a heat exchanger 262 to control the temperature of the feed stream to the distillation tower 264. The feed stream to the distillation tower is the reactor product stream 234. The distillation tower 264 and its preparatory heat exchanger 262 may be configured based on factors such as the properties of the reactor product stream 234 and the desired compositions and properties of the first recycle stream 222 and the separator product stream 224. As illustrated, the distillation system 260 of FIG. 2B includes a reboiler apparatus 266 and a reflux apparatus 268, which may be implemented according to conventional distillation system technologies. Implementations of the present systems and methods, when including a gas/liquid phase separation system, may select systems according to FIG. 2A, to FIG. 2B, or any other suitable gas/liquid phase separation system.

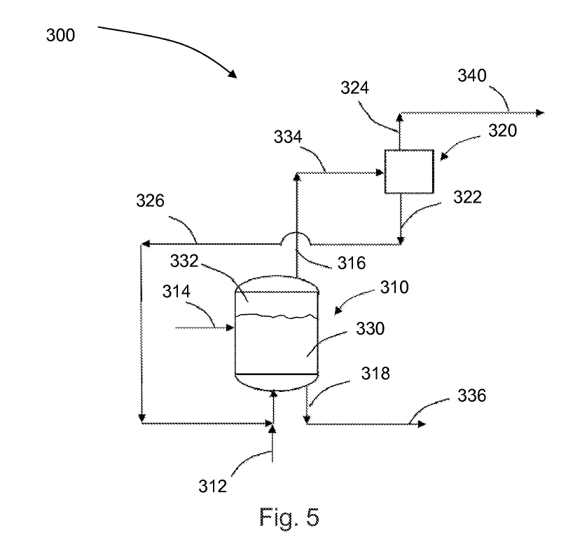

FIG. 3 illustrates yet another schematic illustration of an oligomerization system 300 within the scope of the present disclosure. The oligomerization system 300 of FIG. 3 includes each of the components found in the oligomerization system 200 of FIG. 2, including the gas/liquid phase separation system. While the principles and teachings discussed above apply to the implementation of FIG. 3, it should be recognized that the gas/liquid phase separation system 320 is implemented in a somewhat different manner. More specifically, it should be noted that the gas/liquid phase separation system 320 is implemented so that the liquid phase therefrom forms the first recycle stream 322 and the gas phase therefrom forms the separator product stream 324. Despite the relatively minor change in the configuration of the oligomerization system 300, it is illustrated here to emphasize the diversity of systems and methods that may implemented within the scope of the present disclosure.

More specifically, the oligomerization system 300 is illustrated with an alternative gas/liquid phase separation system 320 to illustrate that a variety of diluents may be utilized in the methods and systems of the present disclosure. In implementations where the diluent is heavier, or has a higher boiling point, than the desired oligomer product, the gas/liquid phase separation system 320 may be configured to separate the unreacted monomer and desired oligomer in the separator product stream 324 and to recycle the diluent in the first recycle stream 322. Such an implementation may be suitable when the separator product stream is fed to a polymerization process that utilizes both the unreacted monomer, such as ethylene, and the desired oligomer, such as 1-hexene. Additionally or alternatively, such an implementation may be suitable when the separator product stream is directed to further separation systems to separate the desired oligomer from the unreacted monomer. In the interest of clarity and conciseness, the remaining elements of FIG. 3 are referenced with numerals corresponding to the analogous elements in FIG. 2 without the need to discuss each element and their relationships and operations anew.

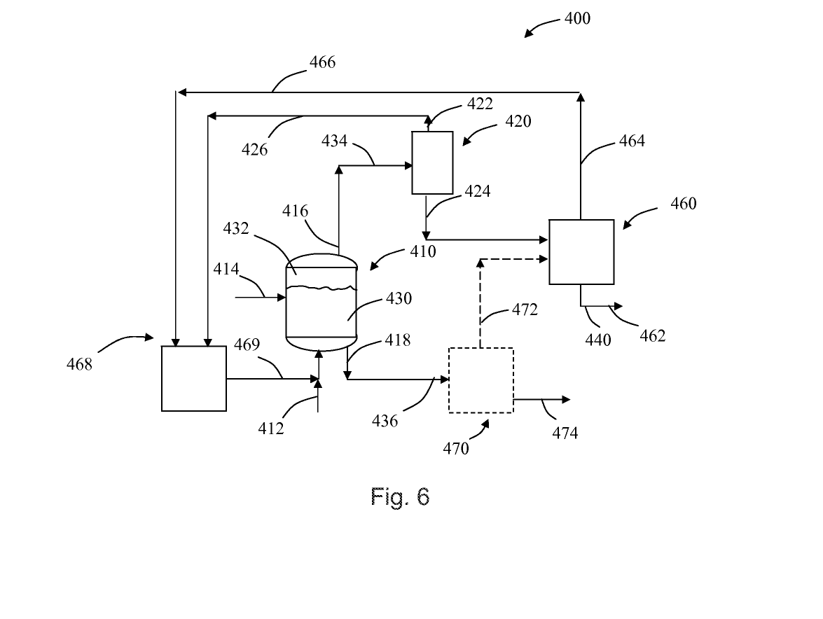

FIG. 4 provides yet another example of a system adapted for implementation of methods within the scope of the present disclosure. As with the foregoing systems, the selection and arrangement of the component parts for implementation of the methods herein distinguishes the present oligomerization systems from those previously developed. The oligomerization system 400 is adapted to include additional separation systems compared to the oligomerization systems 200, 300 of FIGS. 2 and 3. The additional separations systems, and indeed all of the primary equipment of the oligomerization system 400 are illustrated in schematic form to emphasize the diversity of manners in which the systems may be constructed and operated while implemented the methods of the present disclosure.

The oligomerization system 400 includes the oligomer synthesis reactor 410 and the other central elements illustrated and discussed in connection with FIG. 2. Accordingly, analogous component parts of the oligomerization system 400 are referenced by numerals corresponding to those reference numerals used in connection with FIG. 2. In the interest of conciseness, only those elements that are added or that are different between FIG. 2 and FIG. 4 will be described in detail. For the remainder, one of ordinary skill will be able to apply the foregoing discussion of FIG. 2 to the implementation and methods of FIG. 4.

As discussed in connection with FIG. 2, the separator product stream 224 or at least a portion thereof, shown as separator product stream 424 of FIG. 4, may be utilized as the oligomer product stream 240. The oligomerization system 400 of FIG. 4 illustrates another possible use for the separator product stream 424. Specifically, the separator product stream 424 is directed to a diluent recovery system 460. The diluent recovery system 460 may include any suitable separation system adapted to separate the desired oligomer product from any diluent and/or unreacted monomer that may be present in the separator product stream 424. As illustrated, the diluent recovery system 460 forms a concentrated oligomer product stream 462 and a second recycle stream 464. As with the comparison between FIG. 2 and FIG. 3, the concentrated oligomer product stream 462 and the second recycle stream 464 may be either the liquid phase or the gas phase depending on the compositions being separated.

Regardless of the specific equipment implemented in the diluent recovery system, the concentrated oligomer product stream 462 may be utilized as the oligomer product stream from the oligomerization system 400. Additionally, the second recycle stream 464 recycles diluent and unreacted monomer to the oligomer synthesis reactor 410, which may be accomplished through an additional recycle loop 466. As illustrated in FIG. 4, the second recycle stream 462 is blended or mixed with the first recycle stream 422 in a recycle mixer 468 to produce a blended recycle stream 469 that is directed to the oligomer synthesis reactor 410. The recycle mixer 468 may be considered part of the recycle loop 426, the additional recycle loop 466, or as an independent component part of the oligomerization system 400. Additionally, it should be noted that the recycle mixer 468 may be implemented in any suitable configuration desired by an operator. Still additionally, it should be noted that the recycle mixer 468 is representative of the plurality of other processes and equipment that may be applied to the recycle stream(s) preparatory to entering the oligomer synthesis reactor 410.

The oligomerization system 400 of FIG. 4 further illustrates the optional inclusion of a byproduct separation system 470. The byproduct separation system 470 is adapted to receive the reactor byproduct stream 436 and to produce an oligomer-rich stream 472 and a purge stream 474. As will be understood from the discussion above and from the Examples herein, the reactor byproduct stream 436 may include one or more valuable components in addition to undesirable components. For example, the reactor byproduct stream 436 may include diluent, unreacted monomer, and desired oligomer product, in addition to byproduct polymers and catalyst. The byproduct separation system 470 may be adapted to separate the byproduct polymers and catalyst from the remaining components. Accordingly, the oligomer-rich stream 472 may include oligomer, monomer, and/or diluent depending on the composition of the liquid phase effluent 418 from the reactor 410. While any suitable byproduct separation system 470 may be selected and implemented by an operator, one exemplary system is disclosed in pending PCT Application Number PCT/US2010/26661, entitled “System and Method for Selective Trimerization.”

The oligomer-rich stream 472 is illustrated as being directed to the diluent recovery system 460. Additionally or alternatively, the oligomer-rich stream 472 may be processed or recovered in other ways. When the diluent recovery system 460 and the byproduct separation system 470 are both implemented in the oligomerization system 400, the diluent recovery system 460 can be utilized to separate the desired oligomer product from both the separator product stream 424 and the oligomer-rich stream 472. It will be recognized that the compositions of both streams will be generally similar (monomer, diluent, and oligomer) and thus may be concurrently separated relatively easily. The properties (e.g., state, temperature, pressure, etc.) of the separator product stream 424 and the oligomer-rich stream 472 may be different, which may affect the design and/or operation of the diluent recovery system. In some implementations, the combination of the two streams having different properties may facilitate the desired separation in the diluent recovery system, even if requiring additional equipment or parts, such as mixers.

As can be understood from the foregoing discussion, the systems of the present disclosure may be implemented in multiple, operation-specific configurations, with common schematic layouts. Accordingly, the exemplary systems of FIGS. 2, 2A, 2B, 3, and 4 illustrate representative variations on the underlying themes of the present disclosure. One of ordinary skill will be readily able to design and build systems according to the description above utilizing individual component parts optimized for the particular operation. All such operation-specific implementations are intended to be within the scope of the present disclosure, invention, and claims.

One of ordinary skill will recognize the common theme of the various systems resides in the implementation of a method for preparing oligomers from monomers. In its simplest description the methods of the present disclosure include providing an oligomer synthesis reactor; feeding a monomer, a catalyst, and a diluent to the oligomer synthesis reactor; oligomerizing the monomer in the oligomer synthesis reactor in the presence of the catalyst under reaction conditions to produce a desired oligomer product; and utilizing at least a portion of a vapor phase effluent from the reactor as an oligomer product stream. The oligomer synthesis reactor produces a vapor phase effluent and a liquid phase effluent. The vapor phase effluent comprises unreacted monomer, oligomer product, and diluent. The liquid phase effluent comprises catalyst and diluent. As can be understood from the foregoing description of the systems and methods, additional or alternative methods include additional separation steps on one or more of the streams exiting the reactor.

The methods of the present disclosure may include operating the reactor or other parts of the system, including adjusting feed compositions and rates and effluent rates to control the reaction conditions. For example, the catalyst composition, the diluent, the monomer feed, etc., may be selected together with reactor conditions so that the catalyst exhibits a selectivity of at least 90% to the desired oligomer product. In preferred implementations, the catalyst would exhibit an olefin selectivity of at least 95% to the desired oligomer product. In most situations, the desired oligomer product will be an alpha-olefin oligomer.

In some implementations, the monomer feed to the reactor, such as through monomer feed stream 212, may be greater than about 99 wt % ethylene. Depending on the requirements of the operation, the monomer feed stream may have a lower purity requirement for the monomer or the monomer may be a different olefin. In some implementations the monomer feed may be greater than 75 wt % monomer, greater than 80 wt % monomer, greater than 90 wt % monomer, or greater than 95 wt % monomer.

The diluent may be selected based on operating conditions in the oligomerization system and/or based on downstream conditions, such as where product or purge streams may be used. In some implementations, the diluent may be selected from the group consisting of 1-butene, 1-hexene, 1-octene, toluene, propane, butane, isobutane, pentane, isopentane, heptane, octane, nonane, decene, and combinations thereof. As discussed above, the diluent may be selected based at least in part on the boiling point of the diluent relative to the desired oligomer product, such as to facilitate separation operations. In preferred implementations, the diluent may be selected from isobutane, isopentane, heptane, and combinations thereof. In particularly preferred implementations, the diluent may be isobutane.

In some implementations, the methods may be adapted based at least in part of the induction period of the selected catalyst. For example, the reaction temperature and pressure may be controlled to between about 50° C. and about 150° C. and between about 150 psi (10.5 kg/cm2) and about 900 psi (63.3 kg/cm2), with a catalyst reaction residence time of from about 30 minutes to about 6 hours. Alternatively, the induction period of the catalyst may be longer than 30 minutes and the reactor may be controlled to provide a catalyst reaction residence time of between about 60 minutes and about 6 hours. As described above, the systems and methods of the present disclosure provide for two distinct residence times; one for the vapor phase effluent and one for the liquid phase effluent. As used herein, the catalyst reaction residence time refers to the residence time of the liquid phase, which may be calculated by dividing the volume of the liquid phase region by the volumetric flow rate of the liquid phase effluent.

Control of the catalyst reaction residence time may allow an operator to maximize the catalyst utilization rate. Similarly, control of the vapor phase residence time may allow the operator to enhance the selectivity of the oligomerization reaction and to drive more of the desired oligomer product to the vapor phase. For example, using a diluent with an affinity to the oligomer product and with a lower boiling point may carry more of the oligomer product into the vapor phase. For example, isobutane may be preferred in some implementations for these reasons. In implementations that include a gas/liquid phase separation system, the separator product stream may comprise greater than 50% of the oligomer product exiting the oligomer synthesis reactor, in both the vapor phase effluent and the liquid phase effluent. Accordingly, it should be understood that the diluent may be selected based at least in part on the desired oligomer product. Moreover, the monomer feed rate, the vapor phase effluent flow rate, and the liquid phase effluent flow rate may each be controlled to increase the ratio of desired oligomer product in the separator product stream to total desired oligomer product exiting the oligomer synthesis reactor.

In implementations that include both a gas/liquid phase separation system and a byproduct separation system, the monomer feed rate, the vapor phase effluent flow rate, and the liquid phase effluent flow rate may each be controlled to increase the ratio of desired oligomer product in the separator product stream to desired oligomer product in the concentrated oligomer product stream. Effectively, while it is expected that oligomer product will exit the reactor in the liquid phase effluent to be recovered in the concentrated oligomer product stream, in some implementations it is preferred to have a greater portion of the produced oligomer exiting the reactor through the vapor phase effluent. Accordingly, the operating conditions can be controlled in an effort to increase the ratio of oligomer in the vapor phase effluent relative to the liquid phase effluent. As will be seen in the examples below, comparing the mass of oligomer in the separator product stream and in the concentrated oligomer product stream, which may be the two streams entering a diluent recovery system, is one method of comparing the distribution of oligomer between the vapor phase effluent and the liquid phase effluent. In a simple weight ratio of oligomer in each of these streams, the ratio may be greater than about 0.10, greater than about 0.25, greater than about 0.50, greater than about 0.75, or optimally greater than about 1.0. As will be understood, the oligomer synthesis reactor preferentially drives a greater proportion of the oligomer to the vapor phase effluent compared to the liquid phase effluent when the ratio is greater than 1.0. As will be seen below in the examples and alluded to above, the ability to drive more of the desired oligomer product to the vapor phase effluent, and thus to the separator product stream, may depend in part on the monomer selected, the oligomer desired, and the diluent utilized.

### EXAMPLES

### Example 1

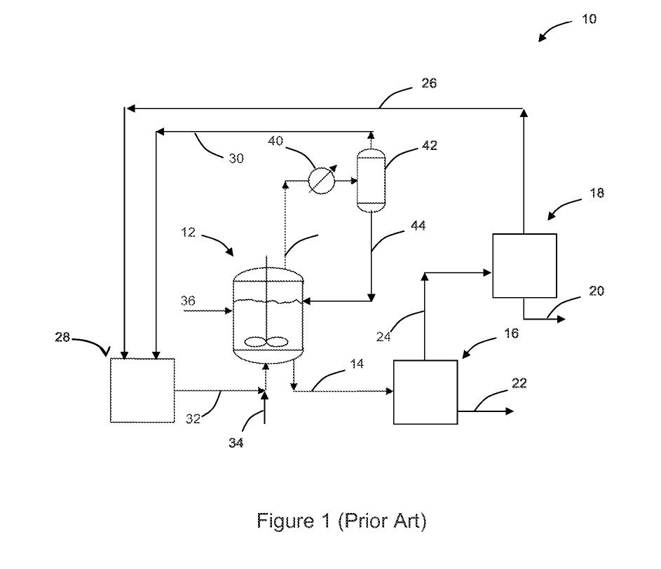

FIG. 1 represents the schematic oligomerization system and method used as a comparative example relative to the systems and methods of the present disclosure. As described above, FIG. 1 is believed to represent the closest systems and methods known or practiced by ordinary skill in the art at the time of this disclosure. As the system was described generally above, the description of this Example 1 will be limited to the specifics of compositions, rates, and other properties. In this Example 1, the systems and methods were adapted for the production of 1-hexene through trimerization of ethylene and were simulated using Simulation Sciences Pro/ii Software, available from Invensys, Inc. In general, the systems were simulated with 15 lbs/hr production of 1-hexene. The stream summaries from the simulation are shown in Table 1 below. For simplicity, the oligomerization reaction and other reactions producing unwanted byproducts are not simulated here. However, it is generally accepted that the rate of unwanted byproduct formation will increase as the concentration of oligomer product increases in the reactor.

TABLE 1

| | Stream Name | | | | | | | | | |
|---|---|---|---|---|---|---|---|---|---|---|
| | 34 | 32 | 38 | 14 | 30 | 44 | 24 | 22 | 20 | 26 |
| Temperature (F.) | 100 | 84 | 150 | 150 | 60 | 60 | 300 | 300 | 300 | 150 |
| Pressure (PSIG) | 55 | 55 | 250 | 250 | 250 | 250 | 75 | 75 | 55 | 55 |
| Liquid Wt Fraction | 0.00 | 0.18 | 0.00 | 1.00 | 0.00 | 1.00 | 0.00 | 1.00 | 1.00 | 0.44 |
| Total Molar Rate | 0.5 | 3.8 | 3.3 | 1.5 | 2.5 | 0.8 | 3.2 | 0.0 | 1.9 | 1.3 |
| (LB-MOL/HR) | | | | | | | | | | |
| Total Mass Rate | 15.0 | 159.2 | 121.0 | 100.0 | 74.4 | 46.6 | 269.1 | 0.9 | 184.3 | 84.8 |
| (LB/HR) | | | | | | | | | | |
| Total Actual Density | 0.04 | 0.08 | 0.23 | 4.63 | 0.22 | 4.86 | 0.14 | 4.57 | 4.66 | 0.16 |
| (LB/GAL) | | | | | | | | | | |

Total Weight Comp. Fractions

| | | | | | | | | | | |
|---|---|---|---|---|---|---|---|---|---|---|
| ETHENE | 1.00 | 0.47 | 0.62 | 0.08 | 0.90 | 0.17 | 0.03 | 0.00 | 0.00 | 0.09 |
| 2MBUTANE | 0.00 | 0.53 | 0.35 | 0.77 | 0.10 | 0.76 | 0.29 | 0.11 | 0.00 | 0.91 |
| HEXENE1 | 0.00 | 0.00 | 0.03 | 0.15 | 0.00 | 0.07 | 0.06 | 0.04 | 0.08 | 0.00 |
| HEPTANE | 0.00 | 0.00 | 0.00 | 0.00 | 0.00 | 0.00 | 0.63 | 0.85 | 0.92 | 0.00 |

Total Weight Comp. Rates (LB/HR)

| | | | | | | | | | | |
|---|---|---|---|---|---|---|---|---|---|---|
| ETHENE | 15.0 | 74.7 | 75.2 | 7.6 | 67.1 | 8.1 | 7.6 | 0.0 | 0.0 | 7.6 |
| 2MBUTANE | 0.0 | 84.3 | 42.4 | 77.3 | 7.1 | 35.3 | 77.2 | 0.1 | 0.0 | 77.2 |
| HEXENE1 | 0.0 | 0.2 | 3.4 | 15.1 | 0.2 | 3.2 | 15.0 | 0.0 | 15.0 | 0.0 |
| HEPTANE | 0.0 | 0.0 | 0.0 | 0.0 | 0.0 | 0.0 | 169.2 | 0.8 | 169.2 | 0.0 |

As can be seen from Table 1, gaseous reactor top stream 38 and reactor bottoms stream 14 exiting the reactor 12 each contain a large quantity of 1-hexene when measured in mass flow rates (data highlighted for convenience). However, as also seen in Table 1, the reactor bottoms stream 14 has a significantly higher concentration of 1-hexene.

### Example 2

Example 2 was developed in much the same manner as Example 1. For example, the data was generated using the same simulation program and the same 15 lbs/hr production rate of 1-hexene. The primary difference between Example 1 and Example 2 is that Example 2 was run utilizing the systems and methods of the present disclosure. More specifically, Example 2 simulated the implementation of the system schematically illustrated in FIG. 4, including the gas/liquid phase separation system, the diluent recovery system, and the byproduct separation system.

In the exemplary simulation of Example 2, the ethylene makeup stream 412 is combined with the combined recycle stream 469 comprising ethylene and diluent and is fed to the reactor 410. The selected diluent for this Example 2 is 2 MButane (isopentane). Catalyst and activator are added to the reactor and the exothermic reactions occur in the reactor. A temperature of 150° F. (65.56° C.) and pressure of 250 psig (17.58 kg/cm²) is maintained by evaporative cooling and some form of direct temperature control.

The vapor phase effluent 416 is withdrawn from the reactor 410, and is cooled to 60° F. in a heat exchanger en route to a separator, which together comprises the gas/liquid phase separation system 420. Because the vapor phase effluent 416 is gaseous, it is substantially free of the non-volatile catalyst components. Uncondensed ethylene, diluent, and a small amount of the desired oligomer product, 1-hexene, exit the gas/liquid phase separation system 420 as a vapor in the first recycle stream 422 and is recycled to the combined recycle stream 469 through the recycle mixer 468. The liquid leaving the gas/liquid phase separation system 420 as separator product stream 424 comprises most of the desired oligomer product, 1-hexene, that was in the vapor phase effluent 416. As a percentage, Table 2 illustrates that greater than 90% of the 1-hexene in the vapor phase effluent leaves the separation system 420 as part of the separator product stream 424. As discussed above, this example is merely one implementation and the portion of the 1-hexene exiting the separation system 420 as separator product stream can be varied by changing the configuration of the separation system. As seen in Table 2, the separator product stream 424 also includes some ethylene and some diluent.

The liquid level is maintained in the reactor by withdrawing liquid phase effluent 418, which comprises catalyst and activator, as well as some 1-hexene, dissolved ethylene, and diluent. The 1-hexene, ethylene, and diluent in the liquid phase effluent 418 are recovered in the byproduct separation system 470 forming the oligomer-rich stream 472. The oligomer-rich stream 472 and the separator product stream 424 are fed to the diluent recovery system 460. In the diluent recovery system 460, the ethylene and the diluent are separated from the oligomer and recycled to the reactor 410 as the second recycle stream 464. The oligomer, the 1-hexene, is then produced as the concentrated oligomer product stream 462. For simplicity, the oligomerization reaction and other reactions producing unwanted byproducts are not simulated here. However, it is generally accepted that the rate of unwanted byproduct formation will increase as the concentration of oligomer product increases in the reactor.

Summaries of each of the streams in FIG. 4 in the simulation of Example 2 are shown in Table 2 below.

TABLE 2

| | Stream Name | | | | | | | | | |
|---|---|---|---|---|---|---|---|---|---|---|
| | 412 | 469 | 416 | 418 | 422 | 424 | 472 | 474 | 462 | 464 |
| Temperature | 100 | 89 | 150 | 150 | 60 | 60 | 300 | 300 | 300 | 150 |
| (F.) | | | | | | | | | | |
| Pressure | 55 | 55 | 250 | 250 | 250 | 250 | 75 | 75 | 55 | 55 |
| (PSIG) | | | | | | | | | | |
| Liquid Wt | 0.00 | 0.01 | 0.00 | 1.00 | 0.00 | 1.00 | 0.00 | 1.00 | 1.00 | 0.02 |
| Fraction | | | | | | | | | | |
| Total Molar Rate | 0.5 | 14.2 | 12.9 | 1.5 | 9.6 | 3.4 | 3.2 | 0.4 | 1.9 | 4.7 |
| (LB-MOL/HR) | | | | | | | | | | |
| Total Mass | 15.0 | 558.1 | 477.7 | 100.0 | 286.3 | 191.4 | 265.5 | 34.5 | 185.2 | 271.8 |
| Rate (LB/HR) | | | | | | | | | | |
| Total Actual | 0.0 | 0.1 | 0.2 | 4.6 | 0.2 | 4.8 | 0.1 | 4.6 | 4.7 | 0.1 |
| Density | | | | | | | | | | |
| (LB/GAL) | | | | | | | | | | |

Total Weight Comp. Fractions

| | | | | | | | | | | |
|---|---|---|---|---|---|---|---|---|---|---|
| ETHENE | 1.00 | 0.54 | 0.61 | 0.08 | 0.90 | 0.17 | 0.03 | 0.00 | 0.00 | 0.15 |
| 2MBUTANE | 0.00 | 0.46 | 0.37 | 0.84 | 0.10 | 0.79 | 0.30 | 0.11 | 0.00 | 0.85 |
| HEXENE1 | 0.00 | 0.00 | 0.02 | 0.09 | 0.00 | 0.04 | 0.03 | 0.02 | 0.08 | 0.00 |
| HEPTANE | 0.00 | 0.00 | 0.00 | 0.00 | 0.00 | 0.00 | 0.64 | 0.87 | 0.92 | 0.00 |

Total Weight Comp. Rates (LB/HR)

| | | | | | | | | | | |
|---|---|---|---|---|---|---|---|---|---|---|
| ETHENE | 15.0 | 298.8 | 291.1 | 7.7 | 257.6 | 33.5 | 7.7 | 0.0 | 0.0 | 41.2 |
| 2MBUTANE | 0.0 | 258.9 | 178.9 | 83.8 | 28.2 | 150.7 | 79.9 | 3.8 | 0.0 | 230.7 |
| HEXENE1 | 0.0 | 0.4 | 7.6 | 8.6 | 0.4 | 7.2 | 7.8 | 0.7 | 15.0 | 0.0 |
| HEPTANE | 0.0 | 0.0 | 0.0 | 0.0 | 0.0 | 0.0 | 170.1 | 29.9 | 170.1 | 0.0 |

A simple comparison of the data in Table 1 and Table 2 reveals that the streams exiting the oligomer synthesis reactor are of similar compositions, at least in that the mass of 1-hexene exiting the reactor is roughly evenly split between the two exiting streams, with the concentration of 1-hexene being higher in the bottoms stream. While the concentration of 1-hexene in stream 418, the liquid phase effluent, is higher than in the vapor phase effluent 416, it should be noted that the flow rates in the two streams are vastly different in both mass flow rates and volumetric flow rates. Accordingly, the total mass of 1-hexene flowing through each of the vapor phase effluent 416 and the liquid phase effluent 418 are comparable.

Additionally, as can be seen in Examples 1 and 2, the present systems and methods provide a comparable residence time for the liquid phase in the reactor while at the same time enabling a lower steady-state concentration of the desired oligomer product, 1-hexene. As shown in Tables 1 and 2, the rate of liquid flow out of the reactor (streams 14 and 418) is 100 lbs/hr. For reactors of equal size, these conditions would lead to equal liquid residence times in both cases. However, the steady state concentration of 1-hexene is significantly reduced from 0.15 wt % to 0.09 wt %. As discussed above, controlling the steady-state concentration of desired oligomer product in the reactor allows greater control over the undesired side reactions and continuing reactions that may occur as the concentration of desired oligomer product increases.

### Example 3

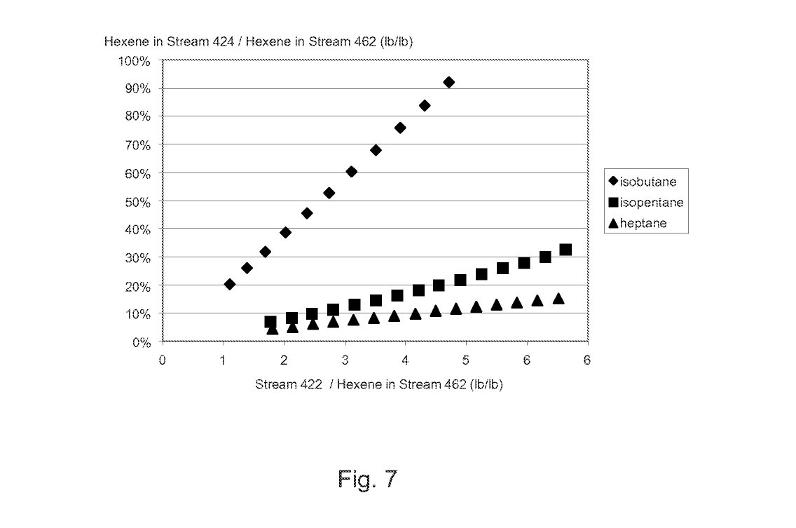

In this Example 3, the simulations of Example 2 were repeated with different diluents to determine potential optimizations. FIG. 5 illustrates the results of varying the diluent selection, the mass flow rate of ethylene, the liquid phase effluent 418 flow rate, and the separator product stream 424 flow rate. For purposes of discussion, the description of FIG. 5 refers to various streams and flow by reference to FIG. 4 and the reference numbers therein. The graph in FIG. 5 illustrates the relationship between two different ratios. The first ratio, illustrated along the conventional y-axis is the ratio of 1-hexene in the separator product stream 424 to 1-hexene in the concentrated oligomer product stream 462. The second ratio, illustrated along the conventional x-axis is the ratio of the first recycle stream 422 to 1-hexene in the concentrated oligomer product stream 462. As can be understood, larger values on the y-axis (i.e., the ratio of 1-hexene in the separator product stream 424 to 1-hexene in the concentrated oligomer product stream) indicates that less of the final product 1-hexene is coming through the liquid phase effluent and through the byproduct separation system 470. In some implementations it may be desirable to minimize the ratio shown on the x-axis to maximize the production rate relative to the recycle rate. The numerator of the ratio on the x-axis may be somewhat fixed by the required production rate while the denominator is a product of the reaction conditions and separation conditions. Minimizing the value on the x-axis while maximizing the value on the y-axis results in a larger quantity of product hexane relative to the required recycle burden of the oligomerization system.

As can be seen from FIG. 5, this Example 3 illustrates that a lighter diluent, such as isobutane, may allow a greater range of control over the oligomerization system. Tables 3-5 below provide the data supporting the graphs of FIG. 5.

TABLE 3

| | | | C6 in | | C6 in | | | | | |
|---|---|---|---|---|---|---|---|---|---|---|
| iso- | 412 | 418 | 418 | 422 | 422 | | C6 in | C6 in | | |
| butane | (lb/hr) | (lb/hr) | (lb/hr) | (lb/hr) | (lb/hr) | 424 | 424 | 462 | y-axis | x-axis |
| 1 | 50 | 59.242 | 11.972 | 16.416 | 0.029 | 74.37 | 3.043 | 15.0 | 20.3% | 1.094 |
| 2 | 55 | 50.113 | 11.139 | 20.684 | 0.041 | 84.243 | 3.876 | 15.0 | 25.8% | 1.379 |
| 3 | 60 | 42.01 | 10.229 | 25.316 | 0.056 | 92.729 | 4.787 | 15.0 | 31.9% | 1.688 |
| 4 | 65 | 34.859 | 9.248 | 30.28 | 0.074 | 99.935 | 5.768 | 15.0 | 38.5% | 2.019 |
| 5 | 70 | 28.564 | 8.207 | 35.533 | 0.097 | 106 | 6.808 | 15.0 | 45.4% | 2.369 |
| 6 | 75 | 23.015 | 7.118 | 41.029 | 0.124 | 111.08 | 7.898 | 15.0 | 52.6% | 2.735 |
| 7 | 80 | 18.103 | 5.989 | 46.724 | 0.155 | 115.33 | 9.027 | 15.0 | 60.2% | 3.115 |
| 8 | 85 | 13.729 | 4.829 | 52.576 | 0.191 | 118.89 | 10.187 | 15.0 | 67.9% | 3.505 |
| 9 | 90 | 9.803 | 3.646 | 58.555 | 0.231 | 121.87 | 11.370 | 15.0 | 75.8% | 3.903 |
| 10 | 95 | 6.245 | 2.444 | 64.629 | 0.275 | 124.4 | 12.571 | 15.0 | 83.8% | 4.309 |
| 11 | 100 | 3.003 | 1.231 | 70.771 | 0.324 | 126.55 | 13.784 | 15.0 | 91.9% | 4.718 |
| 12 | 105 | 0.020 | 0.009 | 76.966 | 0.378 | 128.39 | 15.006 | 15.0 | 100.0% | 5.131 |
| 13 | 110 | 0 | 0.000 | 83.965 | 0.419 | 126.45 | 15.018 | 15.0 | 100.1% | 5.597 |
| 14 | 115 | 0 | 0.000 | 90.964 | 0.461 | 124.5 | 15.018 | 15.0 | 100.1% | 6.063 |
| 15 | 120 | 0 | 0.000 | 97.951 | 0.504 | 122.55 | 15.019 | 15.0 | 100.1% | 6.528 |

TABLE 4

| | | | C6 in | | C6 in | | | | | |
|---|---|---|---|---|---|---|---|---|---|---|
| iso- | 412 | 418 | 418 | 422 | 422 | | C6 in | C6 in | | |
| pentane | (lb/hr) | (lb/hr) | (lb/hr) | (lb/hr) | (lb/hr) | 424 | 424 | 462 | y-axis | x-axis |
| 1 | 50 | 106.61 | 14.006 | 26.518 | 0.064 | 16.94 | 1.009 | 15.0 | 6.7% | 1.768 |
| 2 | 55 | 103.13 | 13.788 | 31.731 | 0.078 | 20.222 | 1.2278 | 15.0 | 8.2% | 2.115 |
| 3 | 60 | 99.66 | 13.562 | 36.944 | 0.093 | 23.488 | 1.4539 | 15.0 | 9.7% | 2.463 |
| 4 | 65 | 96.21 | 13.328 | 42.16 | 0.108 | 26.738 | 1.6877 | 15.0 | 11.3% | 2.810 |
| 5 | 70 | 92.777 | 13.086 | 47.376 | 0.124 | 29.971 | 1.9295 | 15.0 | 12.9% | 3.158 |
| 6 | 75 | 89.362 | 12.836 | 52.594 | 0.140 | 33.184 | 2.1796 | 15.0 | 14.5% | 3.506 |
| 7 | 80 | 85.965 | 12.577 | 57.813 | 0.157 | 36.379 | 2.4384 | 15.0 | 16.3% | 3.854 |
| 8 | 85 | 82.586 | 12.31 | 63.034 | 0.175 | 39.555 | 2.7061 | 15.0 | 18.0% | 4.202 |
| 9 | 90 | 79.228 | 12.033 | 68.256 | 0.194 | 42.709 | 2.9832 | 15.0 | 19.9% | 4.550 |
| 10 | 95 | 75.89 | 11.746 | 73.48 | 0.213 | 45.842 | 3.2699 | 15.0 | 21.8% | 4.898 |
| 11 | 100 | 72.574 | 11.449 | 78.706 | 0.233 | 48.953 | 3.5667 | 15.0 | 23.8% | 5.247 |
| 12 | 105 | 69.279 | 11.142 | 83.933 | 0.254 | 52.042 | 3.8739 | 15.0 | 25.8% | 5.595 |
| 13 | 110 | 66.008 | 10.824 | 89.162 | 0.276 | 55.106 | 4.1919 | 15.0 | 27.9% | 5.944 |
| 14 | 115 | 62.76 | 10.495 | 94.393 | 0.298 | 58.146 | 4.521 | 15.0 | 30.1% | 6.292 |
| 15 | 120 | 59.537 | 10.155 | 99.624 | 0.322 | 61.161 | 4.8618 | 15.0 | 32.4% | 6.641 |

TABLE 5

| | | | C6 in | | C6 in | | | | | |
|---|---|---|---|---|---|---|---|---|---|---|
| | 412 | 418 | 418 | 422 | 422 | | C6 in | C6 in | | |
| heptane | (lb/hr) | (lb/hr) | (lb/hr) | (lb/hr) | (lb/hr) | 424 | 462 | 424 | y-axis | x-axis |
| 1 | 50 | 120.340 | 14.355 | 26.983 | 0.277 | 3.183 | 0.6606 | 15.0 | 4.4% | 1.799 |
| 2 | 55 | 119.790 | 14.238 | 32.032 | 0.327 | 3.777 | 0.7812 | 15.0 | 5.2% | 2.135 |
| 3 | 60 | 119.240 | 14.119 | 37.083 | 0.378 | 4.370 | 0.9009 | 15.0 | 6.0% | 2.471 |
| 4 | 65 | 118.690 | 14.001 | 42.134 | 0.428 | 4.963 | 1.0196 | 15.0 | 6.8% | 2.808 |
| 5 | 70 | 118.140 | 13.884 | 47.185 | 0.478 | 5.555 | 1.1373 | 15.0 | 7.6% | 3.144 |
| 6 | 75 | 117.590 | 13.767 | 52.235 | 0.527 | 6.147 | 1.2541 | 15.0 | 8.4% | 3.481 |
| 7 | 80 | 117.040 | 13.652 | 57.286 | 0.576 | 6.737 | 1.3701 | 15.0 | 9.1% | 3.817 |
| 8 | 85 | 116.490 | 13.537 | 62.336 | 0.625 | 7.328 | 1.4851 | 15.0 | 9.9% | 4.154 |
| 9 | 90 | 115.950 | 13.423 | 67.387 | 0.673 | 7.917 | 1.5992 | 15.0 | 10.7% | 4.490 |
| 10 | 95 | 115.400 | 13.310 | 72.437 | 0.722 | 8.507 | 1.7124 | 15.0 | 11.4% | 4.827 |
| 11 | 100 | 114.850 | 13.198 | 77.487 | 0.769 | 9.095 | 1.8248 | 15.0 | 12.2% | 5.163 |
| 12 | 105 | 114.310 | 13.087 | 82.536 | 0.817 | 9.683 | 1.9362 | 15.0 | 12.9% | 5.499 |
| 13 | 110 | 113.760 | 12.977 | 87.586 | 0.864 | 10.270 | 2.0468 | 15.0 | 13.6% | 5.836 |
| 14 | 115 | 113.220 | 12.867 | 92.635 | 0.911 | 10.857 | 2.1566 | 15.0 | 14.4% | 6.172 |
| 15 | 120 | 112.670 | 12.759 | 97.685 | 0.958 | 11.443 | 2.2655 | 15.0 | 15.1% | 6.508 |

While the data in Tables 3-5 provide the supporting details, FIG. 5 best illustrates the effect that the boiling point of different diluents has on the ability of the present systems and methods to drive more of the desired oligomer product, 1-hexene, to the vapor phase effluent and through to the separator product stream 424. As illustrated in FIG. 5, an operator can drive nearly 100% of the product 1-hexene to the separator product stream by increasing the recycle rate into the reactor (stream 469). An increased recycle rate can be achieved by feeding monomer to the reactor in excess of that needed for the reaction. Additional temperature, pressure, and level controls are needed to maintain all other reactor conditions as the reflux flow rate is increased. Control of these variables in a process such as this can be achieved through several widely-used methods. In some implementations, the ratio illustrated on the y-axis may be greater than about 10%, greater than about 20%, greater than about 25%, or greater than 50%. In more preferred implementations, the ratio illustrated on the y-axis may be greater than 60%, greater than 75%, or even greater than 90%, such as illustrated in Tables 3-5 and FIG. 5.

Specific embodiments of the invention are further described in the following paragraphs:

1. A method for preparing oligomers from monomers, the method comprising:

feeding a monomer feedstream, a catalyst feedstream, and a diluent to the oligomer synthesis reactor;

reacting in the oligomer synthesis reactor the monomer and the catalyst under reaction conditions to produce an oligomer product; wherein the oligomer synthesis reactor produces a vapor phase effluent and a liquid phase effluent; wherein the vapor phase effluent comprises unreacted monomer, oligomer product, and diluent; and wherein the liquid phase effluent comprises catalyst and diluent; and

utilizing at least a portion of the vapor phase effluent as an oligomer product stream.

2. The method of paragraph 1, further comprising:

providing a gas/liquid phase separation system;

passing the vapor phase effluent through the gas/liquid phase separation system to form a first recycle stream and a separator product stream; wherein the first recycle stream comprises diluent and unreacted monomer from the vapor phase effluent; and wherein the separator product stream comprises a majority of the oligomer in the vapor phase effluent; and

utilizing at least a portion of the separator product stream as the oligomer product stream.

3. The method of paragraph 2, further comprising:

providing a diluent recovery system adapted to separate oligomer from diluent and from unreacted monomer;

passing the separator product stream through the diluent recovery system to form a concentrated oligomer product stream and a second recycle stream; wherein the second recycle stream recycles diluent and unreacted monomer to the oligomer synthesis reactor; and

utilizing the concentrated oligomer product stream as the oligomer product stream.

4. The method of paragraph 3, further comprising:

providing a byproduct separation system;

passing the liquid phase effluent through the byproduct separation system to produce an oligomer-rich stream and a purge stream; and

passing the oligomer-rich stream through the diluent recovery system to separate oligomer from diluent and unreacted monomer in the oligomer-rich stream, wherein the separated oligomer is added to the concentrated oligomer product stream, and wherein the diluent and unreacted monomer is recycled to the oligomer synthesis reactor.

5. The method of any one of paragraphs 1-4, wherein the catalyst has a selectivity of at least 901% to the desired oligomer product, and wherein the desired oligomer product is an alpha-olefin oligomer.
6. The method of any one of paragraphs 1-5, wherein the monomer feed is greater than about 99 wt % ethylene.
7. The method of any one of paragraphs 1-6, wherein the diluent is selected from the group consisting of 1-butene, 1-hexene, 1-octene, toluene, propane, butane, isobutane, pentane, isopentane, heptane, and combinations thereof.
8. The method of any one of paragraphs 1-7, wherein said reaction conditions comprise a reaction temperature from about 50° C. to about 150° C., a reaction pressure from about 150 psi (10.5 kg/cm2) to about 900 psi (63.3 kg/cm2), and a reaction residence time from about 30 minutes to about 6 hours.
9. The method of any one of paragraphs 2-8, wherein the gas/liquid phase separation system comprises a cooler and at least one of a flash drum and a distillation column having trays or packing in the vapor zone.
10. The method of any one of paragraphs 2-9, wherein the desired oligomer product is selected from the group consisting of 1-butene, 1-hexene, 1-octene, 1-decene, and mixtures thereof.
11. The method of any one of paragraphs 1-10, wherein the catalyst has an olefin selectivity of at least 95% to the desired oligomer product.
12. The method of any one of paragraphs 1-11, wherein the diluent is lighter than the desired oligomer product, wherein the first recycle stream comprises a gas-phase, and wherein the separator product stream comprises a liquid phase.
13. The method of any one of paragraphs 1-12, wherein the desired oligomer product is 1-hexene, and wherein the separator product stream comprises greater than 50% of the 1-hexene exiting the oligomer synthesis reactor.
14. The method of any one of paragraphs 1-13, wherein the diluent is isobutane.
15. The method of any one of paragraphs 1-14, wherein the diluent is selected based at least in part on the desired oligomer product, and wherein the monomer feed rate, the vapor phase effluent flow rate, and the liquid phase effluent flow rate are controlled to increase the ratio of desired oligomer product in the separator product stream to total desired oligomer product exiting the oligomer synthesis reactor.
16. The method of any one of paragraphs 4-15, wherein the monomer feed rate, the vapor phase effluent flow rate, and the liquid phase effluent flow rate are controlled to increase the ratio of desired oligomer product in the separator product stream to desired oligomer product in the concentrated oligomer product stream.
17. The method of any one of paragraphs 4-16, wherein the ratio of desired oligomer product in the separator product stream to desired oligomer product in the concentrated oligomer product stream is greater than about 10%.
18. The method of any one of paragraphs 4-17, wherein the ratio of desired oligomer product in the separator product stream to desired oligomer product in the concentrated oligomer product stream is greater than about 25%.
19. The method of any one of paragraphs 4-18, wherein the ratio of desired oligomer product in the separator product stream to desired oligomer product in the concentrated oligomer product stream is greater than about 50%.
20. An oligomerization system for preparing oligomers from monomers, the oligomerization system comprising:

an oligomer synthesis reactor; adapted to receive a monomer, a catalyst, and a diluent; and adapted to react the monomer and the catalyst to produce a desired oligomer product and to produce a vapor phase effluent and a liquid phase effluent;

wherein the vapor phase effluent comprises unreacted monomer, oligomer product, and diluent; wherein the liquid phase effluent comprises catalyst and diluent; and wherein at least a portion of the vapor phase effluent is utilized as an oligomer product stream.

21. The system of paragraph 20, further comprising:

a gas/liquid phase separation system adapted to receive the gas phase effluent;

wherein the gas/liquid phase separation system is configured to form a first recycle stream and a separator product stream; and

a recycle loop adapted to recycle the first recycle stream to the oligomer synthesis reactor; wherein the first recycle stream comprises diluent and unreacted monomer; wherein the separator product stream comprises a majority portion of the oligomer product in the gas phase effluent; and wherein at least a portion of the separator product stream is utilized as the oligomer product stream.

22. The system of paragraph 21, further comprising:

a diluent recovery system adapted to receive the separator product stream and to separate oligomer product from diluent and from unreacted monomer to form a concentrated oligomer product stream and a second recycle stream, respectively;

an additional recycle loop adapted to recycle the second recycle stream to the oligomer synthesis reactor; wherein the second recycle stream comprises diluent and unreacted monomer; wherein the concentrated oligomer product stream comprises a majority portion of the oligomer product in the separator product stream; and

wherein the concentrated oligomer product stream is utilized as the oligomer product stream.

23. The system of paragraph 22, further comprising:

a byproduct separation system adapted to receive the liquid phase effluent to produce an oligomer-rich stream and a purge stream;

a purge system adapted to discharge the purge stream from the oligomerization system; and

wherein the oligomer-rich stream is directed to the diluent recovery system; wherein the diluent recovery system is adapted to receive both the separator product stream and the oligomer-rich stream.

All documents described herein are incorporated by reference herein, including any priority documents and/or testing procedures to the extent they are not inconsistent with this text and for all jurisdictions in which such incorporation are permitted. As is apparent from the foregoing general description and the specific embodiments, while forms of the invention have been illustrated and described, various modifications can be made without departing from the spirit and scope of the invention. Accordingly, it is not intended that the invention be limited thereby. Likewise, the term “comprising” is considered synonymous with the term “including” for purposes of Australian law.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

What is claimed is:

1. A method for preparing oligomers from monomers, the method comprising:
feeding a monomer feedstream, a catalyst feedstream, and a diluent to the oligomer synthesis reactor;
reacting in the oligomer synthesis reactor the monomer and the catalyst under reaction conditions to produce an oligomer product; wherein the oligomer synthesis reactor produces a vapor phase effluent and a liquid phase effluent; wherein the vapor phase effluent comprises unreacted monomer, oligomer product, and diluent; and wherein the liquid phase effluent comprises catalyst and diluent; and
utilizing at least a portion of the vapor phase effluent as an oligomer product stream.

2. The method of claim 1, further comprising:
providing a gas/liquid phase separation system;
passing the vapor phase effluent through the gas/liquid phase separation system to form a first recycle stream and a separator product stream; wherein the first recycle stream comprises diluent and unreacted monomer from the vapor phase effluent; and wherein the separator product stream comprises a majority of the oligomer in the vapor phase effluent; and
utilizing at least a portion of the separator product stream as the oligomer product stream.

3. The method of claim 2, further comprising:
providing a diluent recovery system adapted to separate oligomer from diluent and from unreacted monomer;
passing the separator product stream through the diluent recovery system to form a concentrated oligomer product stream and a second recycle stream; wherein the second recycle stream recycles diluent and unreacted monomer to the oligomer synthesis reactor; and
utilizing the concentrated oligomer product stream as the oligomer product stream.

4. The method of claim 3, further comprising:
providing a byproduct separation system;
passing the liquid phase effluent through the byproduct separation system to produce an oligomer-rich stream and a purge stream; and
passing the oligomer-rich stream through the diluent recovery system to separate oligomer from diluent and unreacted monomer in the oligomer-rich stream, wherein the separated oligomer is added to the concentrated oligomer product stream, and wherein the diluent and unreacted monomer is recycled to the oligomer synthesis reactor.

5. The method of claim 1, wherein the catalyst has a selectivity of at least 901% to the desired oligomer product, and wherein the desired oligomer product is an alpha-olefin oligomer.

6. The method of claim 1, wherein the monomer feed is greater than about 99 wt % ethylene.

7. The method of claim 1, wherein the diluent is selected from the group consisting of 1-butene, 1-hexene, 1-octene, toluene, propane, butane, isobutane, pentane, isopentane, heptane, and combinations thereof.

8. The method of claim 1, wherein said reaction conditions comprise a reaction temperature from about 50° C. to about 150° C., a reaction pressure from about 150 psi (10.5 kg/cm2) to about 900 psi (63.3 kg/cm2), and a reaction residence time from about 30 minutes to about 6 hours.

9. The method of claim 2, wherein the gas/liquid phase separation system comprises a cooler and at least one of a flash drum and a distillation column having trays or packing in the vapor zone.

10. The method of claim 2, wherein the desired oligomer product is selected from the group consisting of 1-butene, 1-hexene, 1-octene, 1-decene, and mixtures thereof.

11. The method of claim 10, wherein the catalyst has an olefin selectivity of at least 95% to the desired oligomer product.

12. The method of claim 10, wherein the diluent is lighter than the desired oligomer product, wherein the first recycle stream comprises a gas-phase, and wherein the separator product stream comprises a liquid phase.

13. The method of claim 12, wherein the desired oligomer product is 1-hexene, and wherein the separator product stream comprises greater than 50% of the 1-hexene exiting the oligomer synthesis reactor.

14. The method of claim 13, wherein the diluent is sobutane.

15. The method of claim 12, wherein the diluent is selected based at least in part on the desired oligomer product, and wherein the monomer feed rate, the vapor phase effluent flow rate, and the liquid phase effluent flow rate are controlled to increase the ratio of desired oligomer product in the separator product stream to total desired oligomer product exiting the oligomer synthesis reactor.

16. The method of claim 4, wherein the monomer feed rate, and the liquid phase effluent flow rate are controlled to increase the ratio of desired oligomer product in the separator product stream to desired oligomer product in the concentrated oligomer product stream.

17. The method of claim 16, wherein the ratio of desired oligomer product in the separator product stream to desired oligomer product in the concentrated oligomer product stream is greater than about 10%.

18. The method of claim 17, wherein the ratio of desired oligomer product in the separator product stream to desired oligomer product in the concentrated oligomer product stream is greater than about 25%.

19. The method of claim 18, wherein the ratio of desired oligomer product in the separator product stream to desired oligomer product in the concentrated oligomer product stream is greater than about 50%.

20. An oligomerization system for preparing oligomers from monomers, the oligomerization system comprising:
an oligomer synthesis reactor; adapted to receive a monomer, a catalyst, and a diluent; and adapted to react the monomer and the catalyst to produce a desired oligomer product and to produce a vapor phase effluent and a liquid phase effluent;
wherein the vapor phase effluent comprises unreacted monomer, oligomer product, and diluent; wherein the liquid phase effluent comprises catalyst and diluent; and
wherein at least a portion of the vapor phase effluent is utilized as an oligomer product stream.

21. The system of claim 20, further comprising:
a gas/liquid phase separation system adapted to receive the gas phase effluent;
wherein the gas/liquid phase separation system is configured to form a first recycle stream and a separator product stream; and
a recycle loop adapted to recycle the first recycle stream to the oligomer synthesis reactor; wherein the first recycle stream comprises diluent and unreacted monomer; wherein the separator product stream comprises a majority portion of the oligomer product in the gas phase effluent; and wherein at least a portion of the separator product stream is utilized as the oligomer product stream.

22. The system of claim 21, further comprising:
a diluent recovery system adapted to receive the separator product stream and to separate oligomer product from diluent and from unreacted monomer to form a concentrated oligomer product stream and a second recycle stream, respectively; and
an additional recycle loop adapted to recycle the second recycle stream to the oligomer synthesis reactor; wherein the second recycle stream comprises diluent and unreacted monomer; wherein the concentrated oligomer product stream comprises a majority portion of the oligomer product in the separator product stream; and
wherein the concentrated oligomer product stream is utilized as the oligomer product stream.

23. The system of claim 22, further comprising:
a byproduct separation system adapted to receive the liquid phase effluent to produce an oligomer-rich stream and a purge stream;
a purge system adapted to discharge the purge stream from the oligomerization system; and
wherein the oligomer-rich stream is directed to the diluent recovery system; wherein the diluent recovery system is adapted to receive both the separator product stream and the oligomer-rich stream.
